(19) United States
(12) Patent Application Publication
(10) Pub. No.: US 2020/0316589 A1
(43) Pub. Date: Oct. 8, 2020

(54) A Multi-Well Device for the Processing, Testing, and Multiplexed Analysis of Intact, Fixed, Paraffin or Plastic Embedded (IFPE) Biological Materials

(71) Applicant: Carlos GENTY, Sugar Land, TX (US)

(72) Inventor: Carlos GENTY, Sugar Land, TX (US)

(21) Appl. No.: 16/303,414

(22) PCT Filed: Jan. 4, 2018
(86) PCT No.: PCT/US18/12435

§ 371 Date: Nov. 20, 2018

Related U.S. Application Data
(60) Provisional application No. 62/442,054, filed on Jan. 4, 2017.

(51) Int. Cl. B01L 3/00; B01L 9/00; G01N 1/36; G01N 1/28; G01N 1/31; C12Q 1/6841; G01N 33/68

(52) U.S. Cl. CPC B01L 3/50855; B01L 9/523; G01N 1/36; G01N 1/286; G01N 1/312; C12Q 1/6841; G01N 2001/362; B01L 2300/0822; B01L 2300/0829; B01L 2300/123; B01L 2300/021; G01N 2001/2873; G01N 33/6803

(57) ABSTRACT

The present invention generally relates to methods by which glass, polycarbonate, cyclic olefin polymers (and co-polymers) and other heat and chemical resistant materials (and combinations) are utilized as a multi-well solid support vessel for the processing and testing of intact, fixed, paraffin or plastic embedded (IFPE) biological materials including, but not limited to, tissues, cells, and/or enriched body fluids.

### CROSS-REFERENCE TO RELATED APPLICATIONS

This application claims priority to PCT International Patent Application Number PCT/US2018/012435, filed Jan. 4, 2018, which claims priority to U.S. Provisional Patent Application No. 62/442,054, filed Jan. 4, 2017.

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### SPECIFICATION

### Field of the Invention

The present invention generally relates to methods by which glass, polycarbonate, cyclic olefin polymers (and co-polymers) and other heat and chemical resistant materials (and combinations) are utilized as a multi-well solid support vessel for the processing and testing of intact, fixed, paraffin or plastic embedded (IFPE) biological materials including, but not limited to, tissues, cells, and/or enriched body fluids.

### Background

Multi-well formatted assay plates are commonplace in modern clinical and research laboratories. Multiple published standards for 6, 12, 24, 48, 96, 384 and 1536 well plates have enabled the flourishing of a reagent, consumable and automation ecosystem to support many processing and testing applications ranging from immunological ELISA-like assays to advanced high-content imaging and sequencing assays. Regardless of the analytical technology used, all of these assays share one common trait—that is the ability to screen multiple samples and/or targets in a single, controlled, and automation-compatible batch, thus ensuring the standardization of assay variables from initial sample immobilization, through processing, testing, and analysis. These multi-well systems and the suppliers behind them support the testing of multiple sample matrices such as cells cultures, body fluids, and extracted nucleic acids and proteins from cells, fluids, and tissues.

However, there is a long-standing need in the art for multi-well support systems for the immobilization, processing, testing and analysis of intact, fixed, paraffin or plastic embedded (IFPE) tissue. This current lack of support systems is due in large part to irregularity of the sample sizes that are typically generated by current histotechnology laboratory methods. For instance, the current methods and processes typically begin with tissue excision (or biopsy) and gross dissection followed by sample fixation to arrest autolysis and putrefaction. In order to process the samples for paraffin embedding, the tissue is placed in a tissue cassette (i.e. a perforated container) for processing. The tissue cassette carries the sample forward through dehydration, clearing, and paraffin infiltration, and finally paraffin embedding for orientation, and sectioning (slicing) for immobilization onto a glass microscope slide. The tissue cassette is part of a conventional workflow design system, which was designed to support the creation of microscope slide samples for scientific or medical examination. However, this conventional workflow design system has very significant limitations. With these conventional approaches, sectioning typically occurs on a tissue slicing instrument (e.g. a microtome) where chilled paraffin blocks are loaded and sliced to obtain paraffin tissue sections. Tissue sections are then typically floated onto a warm water bath where a technologist then transfers the tissue onto a microscope slide. This tissue section flotation step is a known source of sample cross-contamination as water baths are typically shared across multiple specimens, which can result in fragments from prior samples contaminating the microscope slides intended for subsequent samples. Once prepared, the microscope slide becomes the final vessel for tissue sample testing for the majority of tests in Anatomic Pathology. However, this testing vessel was designed for microscopic observation and not for the execution of controlled testing where specimen and reagent volumes are precisely managed to ensure analytical precision and reproducibility. In addition, the microscope slide may also serve as the vessel for transport, storage, or for eventual micro-dissection for regions of interest for additional downstream molecular analysis of nucleic acids and proteins—processes that also benefit from enhanced controls enabled by a multi-well vessel compatible with IFPE tissue processing and testing.

The material properties required to support the immobilization, processing, and testing of intact fixed paraffin embedded tissue sections are varied and exacting and very few materials exist that can meet all of the required criteria. Conventional approaches are problematic because a microscope slide, which is typically a glass microscope slide, is required for conventional histological techniques. Also, because the end consumer is a pathologist or investigator viewing a glass slide on a microscope, all engineering controls are centered around this vessel. In fact, using these conventional approaches, current cassette systems are designed and constructed to intentionally limit the size of the tissues for processing and to accommodate the width of a standard microscope slide.

Conventional approaches have been grossly inadequate in the era of high-throughput digital pathology and genomic testing laboratories whose users demand greater precision and improved engineering controls over the process.

Furthermore, with conventional approaches, the cassette system and downstream processing systems have been designed for (and around) the microscope slide and, as a result, have generally been incompatible with multi-well systems that were designed for more flexible matrices such as cells in culture, body fluids, and extracted samples. Therefore, advanced histological testing applications have been limited to the aforementioned microscope slide based approaches.

Effective multi-well support systems for the immobilization, processing, testing and analysis of intact IFPE biological materials are currently non-existent in the market place. There has been a very significant long-felt and unmet need in the art for multi-well support systems for intact IFPE biological materials. The present invention satisfies this long-standing need in the art.

### SUMMARY

The device that this present invention provides for a device and method whereby glass, polycarbonate, cyclic olefin polymers and co-polymers and other heat and chemical resistant materials, and combinations thereof, are deposited in a multi-well solid support vessel for the processing and testing of intact, fixed, paraffin or plastic embedded (IFPE) biological materials (including, but not limited to, tissues, cells, and/or enriched body fluids).

The device is comprised of material properties required to support the immobilization, processing, testing and analysis of intact fixed paraffin or plastic embedded tissue sections in a multi-well system that offers an attractive alternative to single sample slides by offering substantial equivalency to microscope slides (e.g. heat resistance, adhesion, optical clarity, automation compatibility, etc.) with additional improved process controls, a reduced likelihood of contamination and cross-contamination, decreased operator intervention, and compatibility with downstream molecular testing methods. What is more, the present device provides consistent and efficient scoring by whole-well imaging and analysis and the ability to support individual or concurrent performance of several advanced processing and testing analysis methods (e.g. immunofluorescence, immunohistochemistry, ELISA-like methods, etc.). Finally, the present invention allows for barcoded strips, plates, and multi-well assemblies for true positive identification and tracking of samples adding to flexible assay and study designs. All advantages are buttressed by a cost efficiency and flexible automation options that present a clear superiority over current slide-based platforms.

### BRIEF DESCRIPTION OF THE FIGURES

These and/or other aspects and advantages of the invention will become apparent and more readily appreciated from the following description of the embodiments, taken in conjunction with the accompanying figures.

FIG. 1A depicts step 1 of the current state of tissue preparation for intact paraffin or plastic embedded tissue, cells, or biological materials (IFPE) where paraffin or plastic block represents the source of IFPE tissue, cells or biological materials.

FIG. 1B shows step 2 of the current state of tissue preparation for intact paraffin or plastic embedded tissue, cells, or biological materials (IFPE) where a sectioned sample and the resulting paraffin or plastic ribbon is ready for transfer to a flotation bath.

FIG. 1C shows step 3 of the current state of tissue preparation for intact paraffin or plastic embedded tissue, cells, or biological materials (IFPE) where sliced sections (in the form of a ribbon) are placed over the deionized water in the flotation bath, each section is separated with tweezers, and mounted onto a glass slide.

FIG. 1D depicts step 4 of the current state of tissue preparation for intact paraffin or plastic embedded tissue, cells, or biological materials (IFPE) where the microscope slide with immobilized tissue section is ready for processing and testing or region of interest microdissection for downstream molecular techniques.

FIG. 2A depicts step 1 where certain preferred embodiments of the present invention in the form of tissue preparation for standardized and irregular (non-standardized) source IFPE tissue specimens is shown representative of paraffin or plastic blocks depicting the source of IFPE and standardized and irregular (non-standardized) source specimens where a non-standard specimen requires an additional sampling step.

FIG. 2B shows step 2 where certain preferred embodiments of the present invention in the form of tissue preparation for standardized and irregular (non-standardized) source IFPE tissue specimens samples are sectioned (sliced to form a ribbon of sections).

FIG. 2C depicts step 3 where certain preferred embodiments of the present invention in the form of tissue preparation for standardized and irregular (non-standardized) source IFPE tissue specimens tissue sections within the ribbon are separated and individually transferred to specific reservoirs within the multi-well assembly and immobilized onto the bottom surface of the multi-well vessel for processing and testing.

FIG. 3A shows step 1 where certain preferred embodiments of the present invention, showing a representation of multi-well slide assembly, plate strip, and plate layouts with and without holders and a representative high-level testing workflow through prepared multi-well vessels or assemblies (i.e. multi-well slide assembly, a plate strip, and a 96-well molded plate or assembly) are ready for loading or testing after tissue section immobilization is finalized.

FIG. 3B depicts step 2 where certain preferred embodiments of the present invention, showing a representation of multi-well slide assembly, plate strip, and plate layouts with and without holders and a representative high-level testing workflow through multi-well slide assemblies and plate strips are loaded onto a multi-well slide or strip holder (respectively) for processing and testing.

FIG. 3C shows step 3 where the multi-modal processing and testing workflow is described.

### DEFINITIONS

The following definitions are provided for additional clarity, and in accordance with certain preferred embodiments of the present invention.

2D Datamatrix: A barcode font composed of a matrix with two dimensions used to hold data values.

Advanced tissue testing (or staining): Non-routine histological assays that demonstrate molecular targets such as DNA, RNA, or proteins within cells and tissues (i.e. in situ).

Analytical precision: A process for determining how close a group of measurements are to one another. The closer the data replicates, the more likely the results will be similar in the future. Precision is usually calculated and discussed in terms of standard deviations and coefficient of variation (CV). A precise or closely-clustered data set has a smaller CV and is generally more reliable than one that is widely scattered.

Anatomic pathology: A medical specialty that is concerned with the diagnosis of disease based on the macroscopic, microscopic, biochemical, immunologic and molecular examination of organs and tissues.

Autolysis: The destruction of cells or tissues by their own enzymes.

Barcode: A machine-readable code in the form of numbers and a pattern of parallel lines of varying widths.

Biomarker: A measurable substance in an organism whose presence is indicative of some phenomenon.

Biopsy: An examination of tissue removed from a living body to discover the presence, cause, or extent of a disease.

Bright field: The simplest of all the optical microscopy illumination techniques, in which the sample is illuminated from below and observed from above.

Cell culture: Process by which cell are grown under controlled conditions.

Clearing: A histotechnology step within the tissue processing domain where a dehydrant is replaced with a substance that will be miscible with the subsequent embedding medium (e.g. Paraffin). Clearing agents alter the refractive index of tissues rendering them translucent for subsequent microscopic examination.

Coefficient of variation: A measure of relative variability.

Colorimetric: Method of determining the concentration of a chemical element or chemical compound in a solution with the aid of a color reagent.

Cross-contamination: In the context of tissue-based testing, it is the process by which tissue fragments, cells, and other debris are unintentionally transferred from one sample vessel to another.

Cyclic Olefin polymers and co-polymers: An amorphous polymer used in a wide variety of applications including packaging films, lenses, vials, displays, multi-well plates and medical devices.

Dehydration: Removal of water during tissue processing.

DMSO: Dimethyl Sulfoxide.

ELISA-like: Similar to Enzyme-Linked Immunosorbent Assay (e.g. In-Cell Western Blot or Chemiluminescence).

Enriched body fluids: Liquid samples that have undergone a process where specific components (e.g. Cells or nucleic acids) of the liquid sample (e.g. Blood, urine, others) are targeted for capture, isolation, or concentration for testing purposes.

Excision: The act of removing tissue from a patient during surgery.

Extraction: The act of removing a targeted sample from a whole (e.g. DNA extraction from tissue).

Fixation: The preservation of the tissue integrity (morphology) and biochemical composition at a point in time.

Gross dissection: Dissection of relatively large structure and features usually visible with the unaided eye.

High-content imaging: A set of analytical methods that use automated microscopy, multi-parameter image processing, and visualization tools to extract quantitative data from cell populations.

Histotechnology: The scientific discipline that studies organs and tissues of the body including their preparation for viewing under a microscope.

Immobilization: The process by which a tissue section (slice) is mounted onto a glass microscope slide (or other test vessel) for testing.

Immunofluorescence: A light microscopy technique that uses fluorescence microscope and filters for the study and evaluation of nucleic acids and proteins within cells and tissues.

Immunohistochemistry: The process of selectively evaluating antigens that detect specific proteins in the cells of a tissue section by exploiting the principle of antibodies binding specifically to antigens in biological tissues.

in situ Hybridization: A type of hybridization that uses a labeled complementary DNA, RNA or modified nucleic acids strand (i.e. probe) to localize a specific DNA or RNA sequence within cells or a section of tissue.

In-Cell western assay: In-Cell Western (also known as cell based ELISA, in cell western or cytoblot) is an immunocytochemistry method used to quantify target protein or post-translational modifications of the target protein, in cultured cells.

Intact fixed paraffin (or plastic) embedded: Tissues or cells whose structure and morphological context are preserved (fixed) and processed for paraffin or plastic embedding, future testing, and long-term storage. In general testing for specific DNA, RNA, or protein targets performed on such samples is performed in situ (or within the morphological context or location), however constituent cells from these samples may also be micro-dissected for downstream DNA, RNA, or protein extraction and molecular analysis.

Liquid based sample testing: Refers to the sampling, testing, and analysis of non-solid biological tissues and body fluids.

Magnetic beads: Circular nanoparticles used to label antibodies for the detection and measurement of specific analytes or for cell capture and target cell enrichment.

MALDI Mass spectroscopy: The use of matrix-assisted laser desorption ionization as a mass spectrometry imaging technique in which the sample, often a thin tissue section, is moved in two dimensions while the mass spectrum is recorded.

Matrix (or sample matrix): Refers to the components of a sample other than the analyte of interest. In this context, matrix is defined as the intact IFPE biological material, with or without any supporting compounds, that has undergone paraffin or plastic processing prior to immobilization onto the testing vessel.

Mid-western assay: Enzyme-based antigen localization and quantitation procedure for cell and tissue samples performed on microscope slides and read on micro-titer plates.

Multi-modal reader: An instrument capable of performing colorimetric ELISAs, other immunological or biochemical assays, luminescent assays with fluorescence-intensity measurements and in some cases well or slide-based imaging.

Multi-technology: A platform that facilitates sample testing using a variety of detection methods or modalities (e.g. Immunohistchemistry, in situ hybridization, and ELISA-like methods).

Multi-well barrier system: A barrier system that permits the isolation of various portions of a flat testing surface where the sample (or samples) are located.

Multi-well slide assembly: The combination of a multi-well barrier system and the microscope slide that it is attached to.

Multiplexed: A type of assay used in research or clinical laboratories to simultaneously detect and/or measure multiple analytes on a single sample. It is distinguished from procedures that measure one analyte at a time.

Next generation sequencing: Non-Sanger-based, high-throughput DNA sequencing technologies.

Non-standardized: Not in accordance with pre-set and established criteria for inputs to a process. In the context of tissue-based testing, this would describe an embedded tissue sample of irregular shape and dimensions as opposed to a tissue sample prepared to a specific set of dimensions (e.g. 4 mm×4 mm×3 mm) to ensure the best possible outcome after the process is complete.

Nucleic acid: A complex organic substance present in living cells, especially DNA or RNA, whose molecules consist of many nucleotides linked in a long chain.

Optical clarity: The state or quality of being clear or transparent to the eye or analytical instrumentation.

Optical density: The degree to which a refractive medium retards transmitted rays of light.

P-value: The probability for a given statistical model that, when the null hypothesis is true, the statistical summary (such as the sample mean difference between two compared groups) would be the same as or of greater magnitude than the actual observed results.

PCR: Polymerase Chain reaction.

Plate assembly: The combination of plate strips or multi-well slide assemblies and their respective holders representing a batch that is ready for processing and testing.

Polycarbonate: A group of thermoplastic polymers containing carbonate groups in their chemical structures. They are strong, tough materials, and some grades are optically transparent.

Putrefaction: The process of decay or rotting in a body or other organic matter.

Ribbon (of tissue): A group of serial tissue sections that are attached to each other as tissue sectioning (microtomy) occurs. Ribbons are typically separated into their component sections (slices) as they are mounted onto microscope slides or other test vessels.

Sequencing: The process of determining the precise order of nucleotides within a DNA molecule.

Standard deviation: A quantity calculated to indicate the extent of deviation for a group as a whole.

### DETAILED DESCRIPTION OF THE DRAWINGS

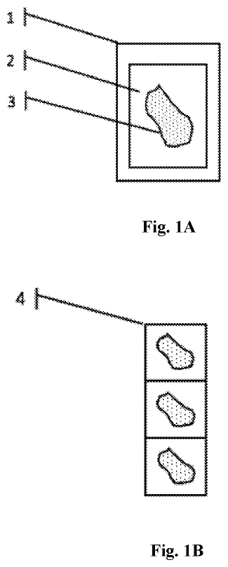

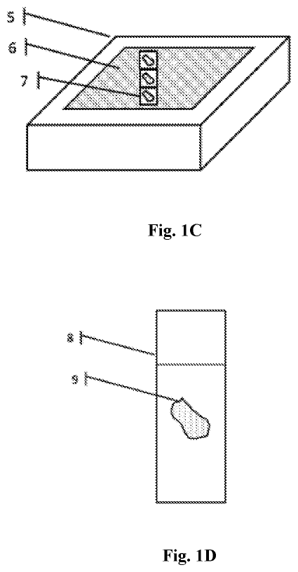

FIG. 1A depicts the current state of tissue preparation for intact paraffin or plastic embedded (IFPE) tissue, cells, or biological material wherein in Step 1 a paraffin or plastic block represents the source of intact paraffin or plastic embedded tissue, cells or biological materials consisting of a tissue cassette 1, solid paraffin or plastic 2, and an irregularly shaped, non-standardized tissue sample 3 that is embedded and supported by and held stationary between tissue cassette 1 and the solid paraffin or plastic 2.

FIG. 1B depicts the current state of tissue preparation for intact paraffin or plastic embedded (IFPE) tissue, cells, or biological material wherein in Step 2 is accomplished where the entire paraffin block step in Step 1 (tissue cassette 1, solid paraffin or plastic 2, and irregularly shaped, non-standardized tissue sample 3) is sliced on a microtome, forming a ribbon of tissue sections 4 for transfer to a flotation bath.

FIG. 1C depicts the current state of tissue preparation for intact paraffin or plastic embedded (IFPE) tissue, cells, or biological material wherein in Step 3 is accomplished by placing (floating) the ribbon of sections 7 over the deionized water 6 in the flotation bath 5, each section is separated with tweezers, and mounted onto a glass slide.

FIG. 1D depicts the current state of tissue preparation for intact paraffin or plastic embedded (IFPE) tissue, cells, or biological material wherein in Step 4 shows a completed tissue slide 8 with now immobilized tissue section 9 is ready for processing and testing or region of interest microdissection for downstream molecular techniques.

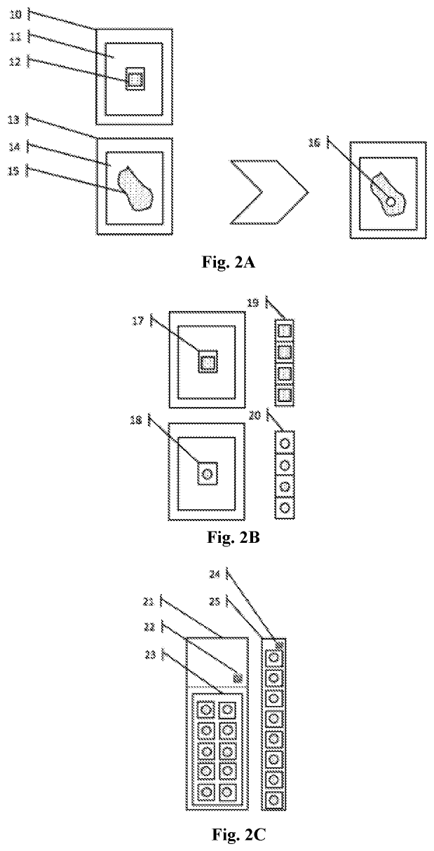

FIG. 2A depicts certain preferred embodiments of the present invention wherein in Step 1 a representative paraffin or plastic block with standardized size and shape of tissue 12 is fixed between a modified tissue cassette 10 for a uniformly shaped sample 12 and solid paraffin or plastic 11 for a standard sample 12 and a block with an irregular and non-standardized size and shape tissue sample 15 which is fixed between a modified tissue cassette 13 for an irregularly shaped, non-standardized sample 15 and solid paraffin or plastic 14 for an irregularly shaped, non-standard sample 15. In the standardized size and shape option, the tissue cassette 10 and solid paraffin or plastic 11 hold the standardized embedded tissue sample (e.g. 4 mm×4 mm×3 mm) 12 in a fixed position in the irregular and non-standardized size and shape option, the tissue cassette 13 and solid paraffin or plastic 14 hold the non-standardized embedded tissue sample 15 in a fixed position. This latter approach requires additional sampling of the irregularly shaped, non-standardized sample 15 by extracting a tissue core (e.g. 2-4 mm diameter) representing a region of interest that is sampled from non-standardized source IFPE Block 16.

FIG. 2B depicts a certain preferred embodiment of the present invention wherein Step 2 is accomplished by slicing the IFPE samples to form a ribbon of sections. Unmodified standardized source block sample 17 is sectioned to form a tissue ribbon 19 without any prior modification to the source block sample 17. Re-embedded (extracted) tissue core 16 is sampled from a non-standard source block, re-embedded to form a new paraffin block 18, and sectioned to form a non-standard source tissue ribbon 20.

FIG. 2C depicts a certain preferred embodiment of the present invention wherein Step 3 is accomplished by separating and individually transferring sections to specific reservoirs within the multi-well assembly and immobilizing them onto the bottom surface of the multi-well vessel (i.e. Multi-well glass slide assembly 21 with multi-well barrier 23, and 2D assembly barcode 22 or multi-well plate strip 25 and 2D plate strip barcode 24 for processing and testing. While irregularly shaped, non-standardized sample 15 tissue core sections are shown (via round tissue), the process is identical for a standard sample 12 tissues (square 4 mm×4 mm×3 mm) that do not require sampling.

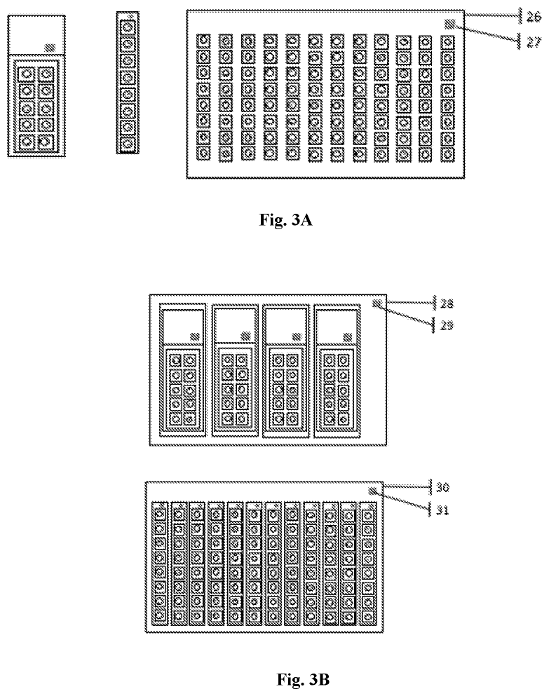

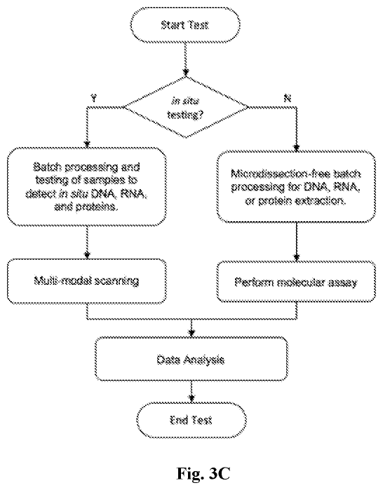

FIG. 3A depicts certain preferred embodiments of the present invention wherein Step 1 shows a representation of multi-well slide assembly, plate strip, and 96-well plate or assembly 26 are depicted that are ready for loading or testing after tissue section immobilization is finalized. The multi-well slide assembly and multi-well plate strip are previously described in FIG. 2C and a 96-well molded plate 26 and 2D barcode 27 are represented.

FIG. 3B depicts certain preferred embodiments of the present invention wherein Step 2 is accomplished by loading multi-well slide assemblies and plate strips onto a multi-well slide or strip holder (respectively) for processing and testing—multi-well assemblies loaded onto a multi-well slide holder 28 with a 2D barcode 29. In this example the multi-well slide assembly holder holds 4 assemblies associated to a batch of 40 wells (or potential IFPE sections). Also, shown is the multi-well plate strip holder 30 with a 2D barcode 31 for processing and testing as a 96 well plate. The multi-well plate strip holder may hold 12 multi-well plate strips associated to a batch of 96 wells (or potential IFPE sections) or a multiple or plurality of 96-wells or other like welled apparatus.

FIG. 3C depicts certain preferred embodiments of the present invention wherein in Step 4 is accomplished by the initiation of the multi-modal processing and testing workflow. Once the assemblies with samples are loaded onto holders or once a 96-well plate has been prepared, the process described herein can begin.

### DESCRIPTION OF PREFERRED EMBODIMENTS

According to certain preferred embodiments, the present invention provides methods by which glass, polycarbonate or cyclic olefin polymers (and co-polymers) and other heat and chemical resistant materials (and combinations) are utilized as a multi-well solid support vessel for the processing and testing of intact, fixed, paraffin or plastic embedded (IFPE) biological materials (including, but not limited to, tissues, cells, and/or body fluids).

In a preferred and non-limiting embodiment, the present invention enables the performance of advanced tissue staining assays for IFPE materials on a multi-well system, that includes, but is not limited to, microscope slides or plates with attached sample isolation barriers, multi-well plates, or plate strips with a plurality of wells (e.g. 2, 3, 4, 6, 12, 24, 48, 96, 384 and 1536 wells) for sample processing, testing, scanning (i.e. Brightfield or fluorescent in situ imaging of the sample, ELISA-like measurements such as colorimetric optical density, fluorescence intensity, particle and or magnetic bead detection, mass spectroscopy), and subsequent data analysis.

In a preferred embodiment, the present invention described herein provides methods, systems and platforms for reliably and accurately transferring advanced histological testing techniques performed on routinely processed tissues (including, but not limited to, immunohistochemistry, multiplexed immunofluorescence, and in situ hybridization and emerging slide-based detection methods) onto a multi-well format. The present invention thus enables multiplex capabilities and greater control of processing, testing, and analysis variables whether performed manually or through liquid handler mediated automation and integration with automated scanning and analysis. The methods and systems and platform of the present invention are also compatible with downstream molecular workflows while eliminating the need for single-slide processing and microdissection for DNA or RNA extractions procedures.

According to other preferred embodiments, the present invention provides a low cost and multi-well plate-based methodology, and provides a single and highly versatile platform for tissue, cell and potentially rare cell analyses.

According to other preferred embodiments, the present invention provides technology that is supportive of the analysis of DNA, RNA, and protein on a single platform.

According to other preferred embodiments, the present invention provides technology that is supportive of multi-specimen, multi-target, or multi-technology testing, scanning and analyses.

According to other preferred embodiments, the present invention provides technology that is compatible with many image analysis systems (preserving contextual information) and downstream molecular applications.

According to other preferred embodiments, the present invention provides technology that is efficient by design through process integration and standardization reducing sample utilization, cycle time, and process variation.

In certain preferred embodiments, the present invention provides methods by which glass, polycarbonate or cyclic olefin polymers (and co-polymers) and other heat and chemical resistant materials are utilized as a multi-well solid support vessel for the processing and testing of intact IFPE biological materials. Preferably, one or more of the polycarbonate, cyclic olefin polymers, and/or cyclic olefin co-polymers, or other heat and chemical resistant materials or any combination(s) thereof, can be molded to form a variety of multi-well slide/well assemblies, multi-well vessels, multi-well plates, or multi-well strips, of any desired and suitable size, shape and dimensions. In the case where glass is the support medium for intact tissue sections, silicone rubber, polycarbonate, cyclic olefin polymers, and/or cyclic olefin co-polymers, or glass or other heat and chemical resistant materials or any combination(s) thereof may be used to assemble the multi-well vessel.

According to certain preferred embodiments, to support the processing, testing, and multiplexed analysis of IFPE biological materials, the critical material criteria described in Table 1 are utilized for processing and testing chemistries and subsequent analyses. Additionally, 2-96 well options are utilized for plates or plate assemblies (i.e. multi-well slide or plate assemblies or multi-well strips with appropriate support frame), the wells are of a symmetrical shape that includes, but is not limited to, circular, square, and other similar shapes, each wells has flat bottoms, a low-profile well-height in the range of 1-4 mm and all the multi-well slide assemblies, strips, plates, and holders are pre-barcoded using a 2D Datamatrix barcode font (or similar font) which stores a unique fixed multi-character length identifier in a base-36 (or modified similar high density number format) and may be labeled with solvent and heat resistant labels with a barcode generated by a Laboratory Information Management System (LIMS) integration or other numbering mechanism.

In yet another preferred embodiment strips and/or multi-well slide assemblies are barcoded and assigned to their parent specimen, plates are pre-barcoded to associate them to their parent specimen(s) and/or batch, and plate holders (frames) are pre-barcoded to associate them to a batch of strips and/or multi-well slide assemblies. Moreover, it is preferred that a multi-well plate, multi-well plate vessel, or multi-well strip or slide assembly, is also disposable and cost-effective.

According to preferred embodiment of the invention, preferred plate materials and requirements (as shown, for example, in Table 1) are employed to effectively replace the single specimen microscope slide for the processing, testing, and multiplexed analysis of IFPE biological materials.

TABLE 1
Representative Materials and IFPE Processing and Testing Requirements*

| | Heat | | | | | | | |
|---|---|---|---|---|---|---|---|---|
| | Resistance | Solvent | Matrix | Flat | Optical | Divisible | | Automation |
| Material | (>90° C.) | Resistance | Adhesion** | Bottom | Clarity | format* | Disposable | Compatible |
| Polystyrene (PS) | Low | Low | High | Yes | High | Yes | Yes | Yes |
| Polypropylene | High | High | Low | Yes | Low | Yes | Yes | Yes |
| Glass | High | High | High | Yes | High | No | No | Yes |
| Quartz | High | High | High | Yes | High | No | No | Yes |
| Polycarbonate(PC) | High | Medium | High | Yes | High | Yes | Yes | Yes |
| Cyclic Olefin (Co) Polymers | High | Medium | High | Yes | High | Yes | Yes | Yes |
| (COP/COC) | | | | | | | | |
| Polyvinyl Chloride (PVC) | Low | Low | Unknown | Yes | High | No | Yes | No |
| Glass Bottom w/PS Frame | Low | Low | High | Yes | High | Yes | Yes | Yes |
| Glass Bottom w/Silicone | High | Low | High | Yes | High | Yes | Yes | Yes |
| Frame | | | | | | | | |
| Glass Bottom w/PC Frame | High | Medium | High | Yes | High | Yes | Yes | Yes |

*Represent preferred criteria for IFPE samples
**Matrix is defined as the intact IFPE biological material, with or without any supporting compounds, that has undergone paraffin or plastic processing prior to immobilization onto the testing vessel whose requirements are described in this table.

According to other preferred embodiments of the present invention, polycarbonate, cyclic olefin polymers, cyclic olefin co-polymers, or other heat and chemical resistant materials, and potential combinations thereof, are utilized as the preferred material(s) that meet critical criteria to support fixed paraffin or plastic embedded tissues and cells.

According to other preferred embodiments, the present invention provides methods and processes for effectively performing advanced processing and testing on IFPE materials. Certain representative process steps and representative testing results are shown in Table 2. Polycarbonate, cyclic olefin polymers, cyclic olefin co-polymers, or other heat and chemical resistant materials, and potential combinations thereof are preferred materials that may be used. It is also contemplated that these materials can be successfully used in applications requiring high temperatures (e.g. PCR) and with concurrent solvent resistance (e.g. DMSO).

TABLE 2
Representative Process Steps to Support Advanced Staining Assays

| | | Material Compatability with Requirement | | |
|---|---|---|---|---|
| | | (Yes/No) | | |
| Key Advanced | | | | Glass Bottom |
| Staining Assay Step | Parameter or Requirement | | | Hybrid |
| or Task | Description | Polycarbonate | CoP/CoC | Assemblies |
| 1. Sample/control | Samples are successfully deposited | Yes | Yes | Yes |
| immobilization | into multi-well vessel using aqueous | | | |
| | media - sections remain intact | | | |
| 2. Sample long term | Samples stored at 4° C., | Yes | Yes | Yes |
| cold storage | and −20° C. | | | |
| 3. Deparaffinization | Samples are deparaffinized and | Yes* | Yes* | Yes* |
| | hydrated using established methods. | | | |
| 4. Heat induced sample | Samples are subjected to temperatures | Yes | Yes | Yes* |
| pre-treatment | of 120° C. for 30 min. | | | |
| 5. Standard incubations | Samples are subjected to temperatures | Yes | Yes | Yes |
| | 37° C. for 18 hrs. | | | |
| 6. Cover slipping | Samples can be coverslipped | Yes* | Yes* | Yes* |
| 7. Imaging | Multi-well scanning imaging, ELISA- | Yes | Yes | Yes |
| | like plate readers, and emerging | | | |
| | detection readers. | | | |

*Requires modification of standard methodologies.

The present invention provides a number of very significant and unexpected advantages which include, but are not limited to (1) a high-throughput alternative to individual (single-sample) microscope slides, (2) a reduced likelihood of sample cross-contamination by assuring that each sample has its own well during sample preparation and testing (i.e. a testing environment that controls reagent volumes and fluid exchange processes), (3) substantial equivalency on key requirements currently provided by microscope slides (requirements including heat resistance (˜120° C.), solvent resistance (complete or partial), matrix support (biological sample), adhesion, optical clarity (comparable to a glass microscope slide), a divisible format, disposability, and automation compatibility, (4) improved process control (by way of fixed/constant sample size, fixed and controlled (no spillover) reagent volumes throughout the life of the assay, and volume control over wash steps), (5) decreased operator involvement through multi-well design engineering controls and liquid handling ecosystem supporting standard multi-well layouts, (6) cost efficiency and flexible automation options when compared to current state slide-based platforms, (7) compatibility with downstream molecular testing methodologies without the need for slide-based micro-dissection (extractions can occur with the strip or plate based system), (8) consistent and efficient automated scoring by whole-well imaging and analysis and/or ELISA-like methods (e.g. Colorimetric, chemiluminescent, or other modality available for detection) using multi-modal readers (as opposed to whole slide imaging of tissues which is time & data storage inefficient for advanced testing techniques because the entire slide is scanned as opposed to limiting scans to a standardized sample or region of interest for analysis), (9) assurance of true positive identification and tracking of samples through barcoded strips, multi-well slide assemblies, and plates for LIMS integration can help, (10) flexible assay and study design (demonstrating the ability to concurrently measure a single analyte on multiple samples, concurrently measure multiple analytes on a single sample, and concurrently measure multiple analytes on multiple samples) and (11) a system that supports the individual performance or concurrent performance of several advanced processing and testing and analysis methods, including but not limited to, immunofluorescence followed by image analysis, immunohistochemistry followed by image analysis, cyclic immunofluorescence with image analysis, in situ hybridization (DNA/RNA) followed by image analysis, fluorescent in situ hybridization followed by image analysis, in situ Polymerase Chain Reaction, enzyme histochemistry followed by image analysis, Mid-Western and In-Cell Western assays and variations (e.g. Microtiter Immunoabsorbent Cytochemical ELISA (MICE), Cytoblot, and Quantitative ELISA-Like Immunohistochemistry (QUELI)), pre-processing and extractions for downstream molecular applications (e.g. Next Generation Sequencing, MALDI Mass Spectroscopy, others), and any combination that would combine reagent chemistries of the above listed methodologies.

The present invention also contemplates and provides for modification of designs for multi-well strips, multi-well slide assemblies, and plates, lower (or higher) profile well designs to facilitate microtomy and immobilization, reagent management or scanning and ensure wider adoption and identification of additional materials, polymers, and/or combinations with glass systems—all of which would be readily apparent to one skilled in the art.

### EXAMPLES

The results shown in Table 3 and Table 4 were obtained from studies that further validate the surprising and unexpected advantages of using microscope slide assemblies and polycarbonate, both of which meet key preferred material criteria for the performance of advance tissue staining assays. Note the low Standard Deviation and Coefficient of Variation values in Table 3 and the replicate P-value results in Table 4 on the issue of process control and reproducibility.

TABLE 3
Representative Results of Optical Density (OD)
measurements and replicate Standard Deviation
(SD) & Coefficient of Variation (CV) Values

| Data | Ki-67 | CD8a | CD10 | Bcl-2 |
|---|---|---|---|---|
| OD - Empty Well | 0.033 | 0.034 | 0.033 | 0.032 |
| OD - Neg Control MIgG | 0.342 | 0.302 | 0.285 | 0.271 |
| OD - Neg Control MIgG Mean (Duplicates) | 0.322 | 0.322 | 0.278 | 0.278 |
| OD - Well 1 | 0.472 | 0.846 | 0.507 | 0.991 |
| OD - Well 2 | 0.477 | 0.837 | 0.498 | 1.026 |
| OD - Mean (Duplicates) | 0.475 | 0.842 | 0.503 | 1.009 |
| OD - Final | 0.153 | 0.520 | 0.225 | 0.731 |
| (Less OD - Neg Control MIgG Mean) | | | | |
| SD | 0.004 | 0.006 | 0.006 | 0.025 |
| CV | 0.745 | 0.756 | 1.266 | 2.454 |

TABLE 4
Representative Results with % positive,
OD measurements, replicate P-values

| | Estimated % | Avg OD | P-Value |
|---|---|---|---|
| Antigen/Antibody (Species) | Positive Cells | (Triplicate) | (vs. Neg) |
| Neg Control/Non-immune | 0% | 0.069 | NA |
| IgGs (M) | | | |
| Neg Control/Non-immune | 0% | 0.022 | NA |
| IgGs (R) | | | |
| P53 (M) | <1% | 0.196 | <.0001 |
| IgA heavy chain (R) | 1% | 0.200 | <.0001 |
| Leu-M1 (M) | 5% | 0.251 | <.0001 |
| Mac387 (M) | 5% | 0.323 | .0001 |
| IgM heavy chain (M) | 5% | 0.379 | .0006 |
| Cleaved-Caspase 3 (R) | <1% | 0.383 | <.0001 |
| Mitosin (M) | 5% | 0.416 | <.0001 |
| KP1 (M) | 5% | 0.505 | <.0001 |
| Lambda light chain (R) | 10% | 0.630 | <.0001 |
| Topoisomerase II (M) | 5% | 0.694 | <.0001 |
| IgG heavy chain (R) | 10% | 0.935 | .0004 |
| P27 (M) | 75% | 1.510 | <.0001 |
| CD31 (M) | 10% | 1.596 | <.0001 |
| Kappa light chain (R) | 15% | 1.672 | .0002 |
| Cytokeratin (M) | 5% | 1.760 | <.0001 |
| MIB1 (M) | 20% | 1.932 | .0003 |
| Bcl2 (M) | 80% | 1.993 | .0001 |
| Tubulin (M) | 25% | 2.219 | <.0001 |
| CD3 (R) | 40% | 2.743 | <.0001 |
| CD45 (M) | 95% | 3.019 | <.0001 |
| CD20 (M) | 60% | 3.457 | <.0001 |

According to certain preferred embodiments, the present invention provides novel platforms for the transfer of advanced histologic testing techniques performed on routinely processed tissues (including, but not limited to, immunohistochemistry, multiplexed immunofluorescence, in situ hybridization, or ELISA-like techniques) onto any desired multi-well format. The present invention enables multiplex capabilities of greater density than current-state single slide methods and provides significantly greater control of processing and assay variables (with or without liquid handler mediated automation) and integration with automated image analysis.

Additionally, representative implementations and utilizations that can be accomplished based on the novel systems, methods and platforms in accordance with the present invention may cover a wide range of services including tissue-based research and consulting services to academic investigators, contract research organizations, laboratory reagent suppliers, biotechnology, and pharmaceutical clients research immunohistochemistry, immunofluorescence, and in situ hybridization services (human/animal), multiplexed assays (multi-specimen, multi-target, or multi-technology), clinical tissue-based biomarker testing services for patients, technical support, tissue processing and slide preparation (IFPE), data analysis, laboratory processing, technical and operational consulting, analysis of DNA, RNA, and protein on a single platform, imaging and preparation for downstream molecular applications, end-to-end consultative support and liquid-based sample testing.

The foregoing descriptions of the embodiments of the present invention have been presented for purposes of illustration and description. They are not intended to be exhaustive or to limit the present invention to the precise forms disclosed. The exemplary embodiments were chosen and described in order to best explain the principles of the present invention and its practical application, to thereby enable others skilled in the art to best utilize the present invention.

1. A device comprising glass, polycarbonate, cyclic olefin polymers (and co-polymers), and other heat and chemical resistant materials (and combinations), wherein that device utilized as a multi-well solid support vessel for the isolation of intact, fixed, paraffin or plastic embedded (IFPE) biological materials with a plurality of wells for sample processing, testing, scanning and subsequent data analysis that are of such a preferred material to meet required criteria for immobilization, processing, and assay execution during the performance of advanced tissue testing.

2. The device of claim 1, wherein the IFPE biological materials are tissues, cells or enriched body fluids.

3. The device of claim 1, wherein said multi-well solid support vessel that includes microscope slides or plates with attached sample isolation barriers, plates, or plate strips used to perform advanced staining assays for IFPE biological materials in a plurality of wells ranging from 2, 3, 4, 6, 12, 24, 48, 96, 384 to 1536 or more wells and multiples therewith for sample processing, testing, scanning and data analysis.

4. The device of claim 1, wherein processing, testing, scanning and analysis are accomplished via a low cost and multi-well plate-based methodology to provide a single and highly versatile platform for tissue, cell and potentially rare cell analyses that is likewise supportive of the analysis of DNA, RNA, and protein on a single platform.

5. The device of claim 1, wherein said multi-well solid support vessel is capable of multi-specimen, multi-target, or multi-technology testing, scanning and analyses.

6. The device of claim 1, wherein said device is compatible with many image analysis systems and downstream molecular applications that is efficient by design through process integration and standardization reducing sample utilization, cycle time, and process variation.

7. The device of claim 1, wherein one or more of the polycarbonate, cyclic olefin polymers, and/or cyclic olefin co-polymers, or other heat and chemical resistant materials or any combination(s) thereof, can be molded to form a variety of multi-well slide/well assemblies, multi-well vessels, multi-well plates, or multi-well strips, of any desired and suitable size, shape and dimensions.

8. The device of claim 1, wherein where glass is the support medium for intact tissue sections, silicone rubber, polycarbonate, cyclic olefin polymers, and/or cyclic olefin co-polymers, or glass or other heat and chemical resistant materials or any combination(s) thereof may be used to assemble the multi-well vessel.

9. The device of claim 1, wherein the processing, testing, and multiplexed analysis of IFPE biological materials are conducted in wells are of a symmetrical shape that includes, but is not limited to, circular, square, and other similar shapes, where each wells has a flat bottom, a low-profile well-height and all the multi-well slide assemblies, strips, plates, and holders are pre-barcoded using a 2D Datamatrix barcode font (or similar font) which stores a unique fixed multi-character length identifier in a base-36 (or modified similar high density number format) and may be labeled with solvent and heat resistant labels with a barcode generated by a Laboratory Information Management System (LIMS) integration or other numbering mechanism.

10. The device of claim 1, wherein strips and/or multi-well slide assemblies are barcoded and assigned to their parent specimen, plates are pre-barcoded to associate them to their parent specimen(s) and/or batch, and plate holders (frames) are pre-barcoded to associate them to a batch of strips and/or multi-well slide assemblies that are disposable and cost effective.

11. A method wherein glass, polycarbonate, cyclic olefin polymers (and co-polymers), and other heat and chemical resistant materials (and combinations thereof), are utilized as a multi-well solid support vessel for the processing, isolation, testing, scanning and subsequent data analysis of intact, fixed, paraffin or plastic embedded (IFPE) biological materials capable of multi-specimen, multi-target, or multi-technology testing, scanning and analyses comprising the steps of:
a. preparing standardized size and shape IFPE sample paraffin or plastic block (tissue cassette and solid paraffin or plastic holding embedded tissue) or sampling a tissue core from the irregular and non-standardized size and shape IFPE sample paraffin or plastic block (tissue cassette and solid paraffin or plastic holding embedded tissue) and re-embedding into a new standardized paraffin or plastic block;
b. slicing the IFPE samples to form a ribbon of sections;
c. separating and individually transferring sections to specific reservoirs within the multi-well assembly and immobilizing them onto the bottom surface of the multi-well vessel;
d. designating, either prior to use or contemporaneously during sample transfer, through 2D code adherence, a multi-well glass slide assembly system with a multi-well barrier and 2D barcode assignment for multi-well plate strip, assembly and a 2D barcode adhesion for tracking, processing, testing, and analysis;
e. Loading and testing of sample tissues after tissue section immobilization is finalized.

12. The method of claim 11, wherein systems and multi-well platforms are utilized for reliably and accurately transferring advanced histological testing techniques performed on routinely processed tissues (including, but not limited to, immunohistochemistry, multiplexed immunofluorescence, and in situ hybridization and emerging slide-based detection methods) onto a multi-well format enabling multiplex capabilities and greater control of processing, testing, and analysis variables whether performed manually or through liquid handler mediated automation, sample procurement standardization and integration with automated scanning and analysis techniques.

13. The method of claim 11, wherein systems and multi-well scanning and analysis platform of the present invention are also compatible with downstream molecular workflows eliminating the need for single-slide processing and microdissection for DNA or RNA extractions procedures.

14. The method of claim 11, wherein DNA, RNA, and protein are detected in situ via batch processing within a single platform via multi-modal scanning.

15. The method of claim 11, wherein DNA, RNA, and protein are detected in situ via micro-dissection free batch processing within a single platform via molecular assay.

16. The method of claim 11, wherein single, double, or a plurality or combination of 2D, 3D, magnetic, matrix bar, holographic, or AR code or the like is used for detection, tracking and processing of histological samples.

17. The Method of claim 11, wherein the processing, testing, and multiplexed analysis of IFPE biological materials are conducted in wells are of a symmetrical shape that includes, but is not limited to, circular, square, and other similar shapes, where each wells has flat bottoms, a low-profile well-height and all the multi-well slide assemblies, strips, plates, and holders are pre-barcoded using a 2D Datamatrix barcode font (or similar font) which stores a unique fixed multi-character length identifier in a base-36 (or modified similar high density number format) and may be labeled with solvent and heat resistant labels with a barcode generated by a Laboratory Information Management System (LIMS) integration or other numbering mechanism.

18. The method of claim 11, wherein said multi-well platforms are designed and utilized for the transfer of advanced histologic testing techniques performed on routinely processed tissues (including, but not limited to, immunohistochemistry, multiplexed immunofluorescence, in situ hybridization, or ELISA-like techniques) onto any desired multi-well format where the present invention enables multiplex capabilities of greater density than current-state single slide methods and provides significantly greater control of processing and assay variables (with or without liquid handler mediated automation) and integration with automated image analysis.

19. The method of claim 11, wherein multi-well solid support vessel can be seamlessly integrated into existing systems, platforms and technologies for utilization in tissue-based research and consulting services to academic investigations, contract research endeavors, biotechnology, pharmaceutics, immunohistochemistry, immunofluorescence, in situ hybridization, multiplexed (multi-specimen, multi-target, or multi-technology) assays, clinical tissue-based biomarker testing, patient services, technical support, tissue processing and slide preparation (IFPE), data analysis, laboratory processing, technical and operational consulting, analysis of DNA, RNA, and protein on a single platform, imaging and preparation for downstream molecular applications, end-to-end consultative support and liquid-based sample testing or a combination thereof.
